(19) United States
(12) Patent Application Publication
(10) Pub. No.: US 2024/0130635 A1
(43) Pub. Date: Apr. 25, 2024

(54) LOWER LIMB SLEEVE FOR DETECTING AND OVERCOMING FREEZE OF GAIT

(71) Applicant: Battelle Memorial Institute, Columbus, OH (US)

(72) Inventors: Bryan Schlink, Gahanna, OH (US); Ian W. Baumgart, Columbus, OH (US); Nicholas J. Tacca, Columbus, OH (US); Eric C. Meyers, Columbus, OH (US); Michael Darrow, Missouri City, TX (US); Lauren R. Wengerd, Columbus, OH (US)

(21) Appl. No.: 18/383,587

(22) Filed: Oct. 24, 2023

Related U.S. Application Data
(60) Provisional application No. 63/419,168, filed on Oct. 25, 2022.

(51) Int. Cl. A61B 5/11

(52) U.S. Cl. CPC A61B 5/112; A61B 2562/0219

(57) ABSTRACT

A device is provided for overcoming freezing of gait. Suitably, the device includes a sleeve sized and shaped to be worn on a leg of a human subject, an array of electrodes carried by the sleeve and positioned to make surface contact with a skin of the leg when the sleeve is worn on by the human subject, and a controller in communication with the array of electrodes. The controller is operative to receive muscle activity data from the array of electrodes indicative of muscle activity in the leg, identify an onset of a freezing of gait episode based on the received muscle activity data, and trigger energization of one or more targeted electrodes in response to identification of the onset of the freezing of gait episode to mitigate against the freezing of gait episode.

This application claims priority to U.S. Provisional Patent Application Ser. No. 63/419,168 filed Oct. 25, 2022 and titled “LOWER LIMB SLEEVE FOR DETECTING AND OVERCOMING FREEZE OF GAIT”, which is incorporated by reference in its entirety.

### BACKGROUND

The following relates to the art of neuromuscular electrical stimulation (NMES), functional electrical stimulation (FES), electromyography (EMG) measurement, and/or related arts. It finds particular application in the detection and overcoming of freezing of gait (FoG) and/or other like symptoms, for example, as may be experienced by individuals or subjects suffering from Parkinson's Disease (PD), and according, it will be described herein at times with reference thereto. However, it is to be appreciated that embodiments and/or subject matter disclosed herein may likewise be suitably employed in connection with other similar applications.

PD is a neurodegenerative disorder that can affect an individual's ability to coordinate movement. Globally and nationally, a significant number of individuals suffer from PD, and the treatment of PD can be a considerable economic burden on health care systems and individuals. There is currently no suitable cure for PD. Efforts have been made on developing medication and technologies that may mitigate detrimental symptoms of PD to help improve patients' quality of life.

One example of a debilitating symptom of PD is known as freezing of gait (FoG), a phenomenon in which the patient experiences a brief and sudden absence in their ability to continue walking, despite their intention otherwise. A significant percentage of PD patients experience FoG, and the percentage tends to increase among patients who have lived with the disease for a significant length of time. Patients have been known to describe the sensation of FoG as though their feet have become temporarily glued to the ground. FoG can tend to occur in situations with heightened cognitive or sensory processes during walking, e.g., such as navigating obstacles or turning around a corner. FoG is generally a progressive condition; early signs can include a hesitation when initiating gait, and brief freezing episodes within an otherwise normal, healthy gait pattern. As FoG progresses, patients can experience freezing with greater frequency, and postural instability can become more prevalent, e.g., inducing a loss of balance and/or significant increase in the likelihood of falls. As a result, PD patients can suffer additional complications, including reduced mobility, loss of independence, prolonged hospitalizations, and/or increased mortality.

Various treatments and/or therapies have been developed to address FoG. These treatments and/or therapies aim to reduce the incidence and/or severity of freezing episodes. For example, some pharmacological treatments and some non-pharmacological treatments may benefit some patients to varying degrees, however, such methods have not been found to be broadly effective at sufficiently alleviating FoG across a suitably significant portion of heterogeneous PD patients.

Treatment of FoG with medication can be complex and incomplete. Pharmacological treatments also have the potential of be accompanied by undesired side effects associated with the given medication. One pharmacological treatment for FoG is levodopa, a drug that metabolizes into dopamine in the brain. Levodopa can potentially reduce the severity and/or duration of freezing episodes, though a significant portion of patients still experience lingering signs of FoG, e.g., such as gait initiation failure. For some PD patients, levodopa is simply ineffective, and others may still experience freezing episodes after taking the medication (referred to as “on-state” FoG). Altering the dosage and frequency of medication may alleviate freezing episodes, but it can lead to worsening of other motor symptoms. Other medications, such as apomorphine, botulinum toxin, and acetylcholinesterase inhibitors, have failed to show suitably consistent improvement in PD patients who freeze. In general, some pharmacological treatments can, therefore, be difficult to prescribe effectively to a heterogeneous group of PD patients who regularly experience FoG.

As pharmacological treatments can present certain challenges, some non-pharmacological methods of treatment for FoG have been explored. For example, deep brain stimulation may have potential as a promising therapy due to its ability to act as a neuromodulator, but it can be undesirable in some cases. For example, deep brain stimulation can involve surgical implantation of devices, which may be complicated, risky and/or otherwise undesirable, and some trials have produced mixed clinical and safety outcomes. Visual, auditory, and tactile cueing methods have also been explored. While cueing approaches can be non-invasive, inexpensive and/or simple, some cueing methods can still be undesirable. For example, some cueing methods have shown inconsistent results, particularly in patients with “on-state” freezing. In some instances, cues are provided continuously, which may result in noncompliance and/or habituation. In some cases, cues may not be customized to the individual, contradicting the heterogeneous nature of FoG. Additionally, PD patients who experience FoG may tend to have decreased cognitive and executive functioning compared to PD patients that do not freeze, further making cueing techniques less than desirable in some cases.

Accordingly, in some cases, there can be a desire for a non-pharmacological technology to treat and/or otherwise address FoG, and in particular, for a personalized, automated technique to detect and overcome FoG that is suitably effective across a significantly broad heterogenous group of PD patients. In accordance with some embodiments disclosed herein, a new and/or improved system, device, apparatus and/or method are described which can satisfy such a desire and/or can mitigate against drawbacks, limitations and/or concerns that may be associated with some otherwise conventional and/or previously developed approaches.

### BRIEF SUMMARY

In accordance with some illustrative embodiments disclosed herein, a device is provided for overcoming freezing of gait (FoG). Suitably, the device includes: a sleeve sized and shaped to be worn on a leg of a human subject; an array of electrodes carried by the sleeve and positioned to make surface contact with a skin of the leg when the sleeve is worn on by the human subject; and a controller in communication with the array of electrodes. Suitably, the controller is operative to: receive muscle activity data from the array of electrodes indicative of muscle activity in the leg; identify an onset of a FoG episode based on the received muscle activity data; and trigger energization of one or more targeted electrodes in response to identification of the onset of the FoG episode to mitigate against the FoG episode.

In accordance with some illustrative embodiments disclosed herein, an apparatus is provided that mitigates FoG in a subject with a neurological impairment. Suitably, the apparatus includes: a sleeve arranged to be fitted on a leg of the subject; an array of electrodes carried by the sleeve and positioned to make contact with a skin of the leg when the sleeve is fitted to the subject; and at least one processor which executes computer program code from at least one memory, wherein the at least one memory and the computer program code are configured, with the at least one processor, to cause the apparatus at least to: collect muscle activity data from the array of electrodes, the muscle activity data being indicative of muscle activity in the leg; detect onsets of FoG episodes based on the collected muscle activity data; and trigger energization of one or more targeted electrodes in response to a detection of an onset of a FoG episode to mitigate against the FoG episode.

In accordance with some illustrative embodiments disclosed herein, a method is provided for detecting and overcoming FoG. Suitably, the method includes: monitoring muscle activity of a leg of a subject with an array of electrodes positioned in contact with a skin of the leg, the array of electrodes being carried by a sleeve worn on the leg; identifying an onset of a FoG episode based on the monitored muscle activity; and selectively energizing of one or more targeted electrodes of the array of electrodes in response to identification of the onset of the FoG episode to remediate the FoG episode.

In accordance with some illustrative embodiments disclosed herein, an apparatus is provided that provides walking assistance for a subject with a neurological impairment. Suitably, the apparatus includes: a sleeve arranged to be fitted on a leg of the subject; an array of electrodes carried by the sleeve and positioned to make contact with a skin of the leg when the sleeve is fitted to the subject; and at least one processor which executes computer program code from at least one memory, wherein the at least one memory and the computer program code are configured, with the at least one processor, to cause the apparatus at least to: collect electromyography (EMG) data from the array of electrodes, the EMG data being indicative of muscle activity in the leg; model the EMG data to determine assistive neuromuscular electrical stimulation (NMES) for improving a gait of the subject; and trigger energization of one or more targeted electrodes in accordance with the determined assistive NMES.

Numerous advantages and benefits of the subject matter disclosed herein will become apparent to those of ordinary skill in the art upon reading and understanding the present specification. It is to be understood, however, that the detailed description of the various embodiments and specific examples, while indicating suitable embodiments, are given by way of illustration and not limitation.

### BRIEF DESCRIPTION OF THE DRAWINGS

The following Detailed Description makes reference to the figures in the accompanying drawings. However, some suitable embodiments and/or the subject matter disclosed herein may take form in various components and arrangements of components, and in various steps and arrangements of steps. The drawings are only for purposes of illustrating some suitable embodiments and are not to be construed as limiting. Further, it is to be appreciated that the drawings may not be to scale.

FIG. 1 diagrammatically illustrates a subject wearing an electrode bearing sleeve in accordance with some suitable embodiments disclosed herein.

FIG. 2 diagrammatically illustrates the sleeve of FIG. 1 in accordance with some suitable embodiments disclosed herein.

FIG. 3 diagrammatically illustrates a system incorporating the sleeve of FIG. 1 in accordance with some suitable embodiments disclosed herein.

FIG. 4 diagrammatically illustrated a number of hypothetical graphs referred to for describing some suitable embodiments disclosed herein which address FoG, wherein the horizontal axis of each graph represents time in arbitrary units and the vertical axis of each graph represents a magnitude of the respective values in arbitrary units.

FIG. 5 diagrammatically illustrates a method and/or process of detecting FoG and remediating the same in accordance with some suitable embodiments disclosed herein.

FIG. 6 diagrammatically illustrates a method and/or process of detecting FoG and remediating the same in accordance with some further suitable embodiments disclosed herein.

FIGS. 7 and 8 diagrammatically illustrate examples of closed-loop control approaches employing wearable sensors such as the EMG via electrodes arrays and inertial IMU sensors to calculate joint torque for use as a user-specific control signal.

### DETAILED DESCRIPTION

The following disclosure provides many different embodiments, or examples, for implementing different features of the subject matter discussed herein. Specific examples of components and arrangements are described below to simplify the present disclosure. These are merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Further, spatially relative terms, such as “left,” “right,” “side,” “back,” “rear,” “behind,” “front,” “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

For clarity and simplicity, the present specification shall refer to structural and/or functional elements, relevant standards, algorithms and/or protocols, and other components, methods and/or processes that are commonly known in the art without further detailed explanation as to their configuration or operation except to the extent they have been modified or altered in accordance with and/or to accommodate embodiment(s) presented herein. It will be appreciated that modifications to disclosed and described examples, arrangements, configurations, components, elements, apparatuses, methods, materials, etc. can be made and may be desired for a specific application. In this disclosure, any identification of specific materials, techniques, arrangements, etc. are either related to a specific example presented or are merely a general description of such a material, technique, arrangement, etc. Identifications of specific details or examples are not intended to be, and should not be, construed as mandatory or limiting unless specifically designated as such.

In general, in accordance with some suitable embodiments, there is disclosed herein a system, device, apparatus and/or method that senses and/or detects the onset or potential onset of FoG or the like from real-time or near real-time changes in the muscle activity of a subject's lower limb or leg and timely delivers FES, NMES or the like to selected lower limb or leg muscles affected to overcome and/or inhibit a freezing episode. For example, relative to the detected onset, the FES may be delivered, instantaneously, promptly thereafter, or otherwise in time to overcome and/or inhibit the freezing episode. In some suitable embodiments, a high-density array of dual-purpose electrodes embedded within a sleeve fitted to and/or worn on the subject's lower limb or leg may use EMG or the like to monitor and/or record muscle activity in the subject's lower limb or leg, for example, continuously or near continuously (i.e., periodically and/or intermittently at suitably short measurement intervals) in real-time or near real-time. FES, NMES or the like may also in turn be selectively delivered via selected ones of the electrodes to stimulate selected muscles. In accordance with some suitable embodiments, computer logic or the like (e.g., including advanced or other like machine learning (ML), a neural network (NN), or artificial intelligence (AI) or the like) may identify patterns of altered muscle activity indicative of and/or otherwise associated with an onset (e.g., imminent, potential or otherwise) of a freezing episode, triggering the delivery of stimulation, such as FES, NMES or the like, to selected muscles affected, thereby overcoming and/or inhibiting the freezing episode and allowing the subject to continue an otherwise normal, healthy gait. Advantageously, in some suitable embodiments, the onset of and/or potential for a freezing episode can be identified and intervention applied before the subject's lower limb or leg actually freezes entirely, thus allowing for a continuous uninterrupted gait without a loss of balance or stability.

In some suitable embodiments, a training or learning period may be used to establish a baseline or model representative of a subject's otherwise normal or healthy gait. In practice, this baseline or model may be customized and/or tailored to the specific subject. Suitably, during the training or learning period, the aforementioned sleeve may be fitted to and/or worn by a subject and EMG data obtained therefrom while the subject is engaged in their normal, health gait, and the baseline or model can be generated or established based upon and/or from this collected training data. Accordingly, when the sleeve is in an actual operative or therapeutic mode, recognized patterns or the like in the monitored EMG data, for example, which may substantially or otherwise suitably deviate from the baseline or model or otherwise indicate the onset (e.g., immediate, potential or otherwise) of a freezing episode, can be used to trigger corrective stimulation to selected muscles, for example, via FES, thereby overcoming and/or inhibiting the identified onset of the freezing episode and allowing the subject to continue with their otherwise normal, healthy gait.

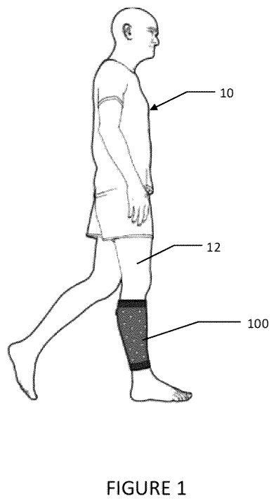

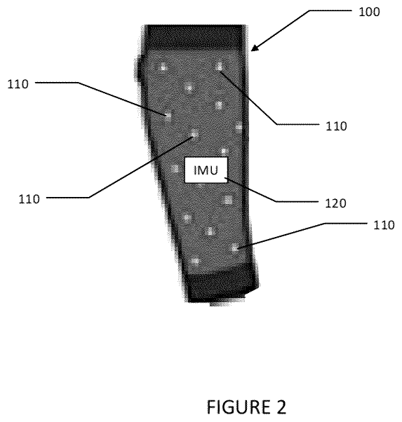

With reference now to FIG. 1, there is shown a subject 10 wearing a suitable sleeve 100, in accordance with some embodiments disclose herein, on their lower limb or leg 12. In practice, the leg sleeve 100 may be constructed of a suitably stretchable and/or flexible fabric or other material that is sized and/or dimensioned to snuggly fit well on the subject's lower limb or leg 12. The sleeve 100, as further illustrated in FIG. 2, may incorporate a high-density array of dual-purpose electrodes 110. Suitably, the high-density array of dual purpose electrodes 110 may be both for selectively obtaining EMG measurements and/or data on one hand and in turn for selectively administering FES, NMES or the like on the other hand. In some suitable embodiments, the high-density array may include a hundred or more electrodes 110. In some suitable embodiments, the high-density array may include as much as 160 electrodes 110 or more. In some suitable embodiments, the high-density array may include less than one hundred electrodes 110.

As shown in FIG. 1, the leg sleeve 100 may be worn on and/or fitted about the subject's calf or lower leg portion. In some alternate embodiments, the leg sleeve may be worn on and/or fitted about the subject's thigh or upper leg portion. In still other alternative embodiments, the leg sleeve 100 may be worn on and/or fitted about both the calf and thigh or both the upper and lower leg portions. In this case, the sleeve 100 may include two separate portions for the two respective leg portions, or alternately, the sleeve 100 may include only a single portion which may extend, for example, over the knee, to encompass both the upper and lower leg portions. Additionally, for simplicity herein, the leg sleeve 100 is shown on only one leg 12 of the subject 10. In practice, a pair of leg sleeves 100 may be worn on and/or fitted about the legs of the subject 10 (i.e., one of the pair for each leg of the subject 10) and their operation coordinated and/or otherwise controlled accordingly.

International applications published under the Patent Cooperation Treaty (PCT) as International Publication No. WO 2022/026821, publication date Feb. 2, 2022, and International Publication No. WO 2022/026177, publication date Feb. 2, 2022, both of which international PCT application publications are incorporated by reference herein in their entirety, describe similar electrode bearing sleeves worn on and/or fit to a subject's upper limb or arm. In some suitable embodiments, the leg sleeve 100 disclosed herein may be similarly constructed and/or provisioned like the arm sleeves (e.g., including any applicable incorporated and/or adjunct components and/or elements, such as, without limitation, the electrodes, etc.) described in the aforementioned international PCT application publications, however, the leg sleeve 100 may be sized, dimensioned, shaped, formed and/or otherwise modified to snuggly fit well and/or be worn on the subject's lower limb or leg 12 as opposed to an arm and/or operated to remediate FoG in PD patients as described herein.

In practice, the leg sleeve 100 is sufficiently snug to the subject's leg 12 to help ensure that the electrodes 110 incorporated in the sleeve 100 make reliable and/or otherwise suitable electrical contact with the skin of the subject's leg 12. High resistivity contact, or intermittent contact, can result in noisy EMG signals. For NMES and/or FES intended to stimulate muscle contractions, the applied NMES and/or FES signal can be large, e.g., on the order of 100-200 volts or higher with corresponding electrical current. Poor and/or intermittent electrical contact between an electrode 110 and the skin at these high voltages can result in electrical arcing that can be painful and/or damaging to the skin.

Suitably, the leg sleeve 100 is sized, dimensioned and/or otherwise formed and/or shaped to encourage accurate, reliable and repeatable positioning of the electrodes 110 at selected and/or desired locations on the subject's leg 12. EMG signal interpretation can be dependent upon accurate mapping of the electrodes 110 to the underlying musculature. Effective NMES and/or FES or other like stimulation of selected musculature can also be dependent on electrode placement. In some suitable embodiments, this can be achieved by a priori knowledge of the mapping. However, if the sleeve positioning on the leg 12 is imprecise or differs from one donning of the sleeve 100 to the next, then this mapping may not be consistent with actual electrode placement. While certain data processing can accommodate for some spatial shift due to imprecise or variable positioning of the sleeve 100, it can be helpful to have the sleeve 100 positioned as accurately and/or as repeatably as feasible. A related concern can be changes in alignment subsequent to donning due to movement of the leg 12 on which the sleeve 100 is fitted. Such movement can result in the positioning of the electrodes 110 relative to the underlying musculature shifting. Accordingly, the sleeve 100 may be made to be suitably elastic, stretchy and/or flexible and/or otherwise dimensioned, sized and/or formed or shaped so as to securely form fit significantly snug to the wearer's leg 12 to encourage accurate and/or repeatably consistent placement and/or minimize shifting during movement, while still remaining suitably comfortable to wear and/or easy to don and/or doff. For example, the sleeve 100 may be made of stretchable spandex fabric, as one nonlimiting illustrative example.

As shown in FIG. 2, in some suitable embodiments, the leg sleeve 100 may further incorporate an inertial measurement unit (IMU) 120 which may detect, sense, measure, record and/or monitor the motion of the wearer's leg 12. For example, the IMU 120 may comprise and/or incorporate one or more accelerometers, gyroscopes, or the like which can sense and/or detect movement of the leg 12 and output a representative signal in response thereto. In practice, data obtained from the IMU 120 may be used to recognize and/or identify (or suitably assist in the recognizing and/or identifying) of where within a gait cycle the subject's leg 12 is at any given time, for example, which phase of the gait cycle the leg 12 may be in. Alternatives to the illustrative IMU 120 are also contemplated for tracking the gait cycle, such as pose estimation derived from video of the subject (i.e., pose estimation computer vision). Accordingly, FES and/or NMES stimulations may be coordinated so that the musculature of the leg 12 (e.g., one or more selected muscles or muscle groups of the leg 12) is suitably stimulated to continue an otherwise normal, healthy gait at the appropriate point in the gait cycle, i.e., depending on where in the gait cycle the leg 12 is when the onset of a freeze episode is detected and/or sensed. In some suitable embodiments, the current phase of the subject's gait may be recognized and/or determined base, at least in part, on measured EMG data obtained from electrodes 110 in the sleeve 100. In some suitable embodiments, the current phase of the subject's gait may be recognized and/or determined base, at least in part, on both measured EMG data and motion data obtained from the IMU 120.

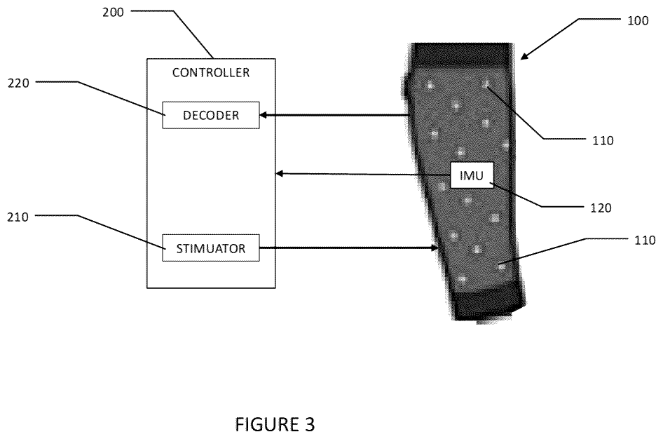

With reference to FIG. 3, in some suitable embodiments, an electronics module or other like hardware controller 200 is provided, for example, which may operate the leg sleeve 100 to perform FES, NMES, and/or readout of EMG and/or IMU measurements. As shown, for FES and/or NMES, the controller 200 may include a stimulator 210 that selectively energizes designated subsets (i.e., one or more) of the electrodes 110 incorporated in the sleeve 100 to execute an appropriate FES and/or NMES of the subject's leg 12. Accordingly, the stimulator 210 may comprise and/or include an electrical generator or power supply, a pulse generator, an amplifier, etc., under the regulation and/or control of the controller 200, to generate and/or produce a desired electrical signal or the like having a suitable waveform and/or magnitude for driving and/or energizing selected one or more of the electrodes 110 in the sleeve 100 to achieve FES and/or NMES of one or more muscles or muscle groups of the leg 12 which map to the location(s) of the selectively energized electrodes 110. In some suitable embodiments, the stimulation can result in contraction of one or more selected muscles or muscles groups corresponding to the selectively energized electrodes 110 thereby leading to and/or urging of an induced movement of the leg 12 which is desired, for example, based on a current phase of the subject's gait which suitably may be recognized and/or determined by the controller 200 in response to and/or accordance with motion data obtained by the controller 200 from the IMU 120 and/or obtained EMG data.

For EMG or other like readout, the controller 200 may selectively read voltages on the electrodes 110 to measure EMG produced by the musculature of the leg 12. For example, as shown in FIG. 3, the controller 200 may comprise and/or incorporate a data processing hardware module or unit or decoder 220 that receives, processes and/or decodes signals and/or data obtained from the high-density array of electrodes 110 in the sleeve 100. An optional EMG amplifier (not shown) may amplify the EMG signals prior being input to the decoder 220 for decoding. In practice, the decoder 220 may employ received and/or obtained EMG data and/or signals from the electrodes 110 of the sleeve 100 to recognize and/or otherwise determined, based at least partially thereon, the onset (e.g., imminent, potential or otherwise) of a freezing episode.

In some suitable embodiments, the controller 200 and/or selected components thereof may be in operative communication with the sleeve 100 and/or the high-density array of electrodes 110 and/or the IMU 120 via wired, wireless or a combination of wired and wireless connections and/or communication channels of suitable bandwidth to accommodate the signals and/or data being exchanged thereover. In some suitable embodiments, some of the electronics or controller hardware or controller components may be integrated into the sleeve 100. In the case of wireless connections and/or communication channels being employed, the controller 200 and/or sleeve 100 may be equipped and/or provisioned with suitable transceivers that may selectively receive and/or transmit suitable signals and/or data wirelessly as appropriate.

International applications published under the Patent Cooperation Treaty (PCT) as International Publication No. WO 2022/026821, publication date Feb. 2, 2022, and International Publication No. WO 2022/026177, publication date Feb. 2, 2022, both of which international PCT application publications are incorporated by reference herein in their entirety, describe electronics modules, drive/control and/or other electronic circuits, circuit boards, etc. (along with adjunct components and/or elements) which are used in conjunction with electrode bearing sleeves to measure and/or collect EMG data or the like and/or selectively administer FES, NMES and/or like stimulations, both via dual-function electrodes incorporated in the sleeve. In some suitable embodiments disclosed herein, the sleeve 100, electrodes 110, IMU 120, controller 200, stimulator 210 and/or decoder 220 disclosed herein may be similarly constructed and/or provisioned, e.g., to include any applicable components and/or elements, for a similar purpose and/or function to like benefit and/or advantage as described in the aforementioned international PCT application publications, however, the same may be altered and/or otherwise modified for the subject's lower limb or leg 12 as opposed to an arm, e.g., to achieve the FoG remediation as disclosed herein.

Wearable devices provide an effective, non-invasive means to monitor and record physiological data from clinical populations with neurological disorders. One of their benefits is that they allow the user or subject 10 to walk/move unencumbered, so data recorded from these devices represent their natural movements. Accordingly, the controller 200 and/or selected components thereof may be implemented in or on a portable or mobile device which can be strapped to and/or otherwise carried or worn by the subject 10 along with the sleeve 100. In some suitable embodiments, data processing and/or control logic or the like may be remotely located and/or provisioned and signals and/or data can be communicated wirelessly with other components worn on or carried by the subject 10. In some embodiments, the sleeve 100 may be provisioned with and/or connected to a local power supply carried by the subject 10 for selectively energizing and/or otherwise driving the electrodes 110 in accordance with control signals and/or data received wirelessly and/or remotely, for example, from the controller 200 and/or stimulator 210.

In some suitable embodiments, the sleeve 100 is sufficiently easy to don and doff and can eliminate or reduce the time which would otherwise have to be taken to manually place electrodes, while still allowing for a high-density array contain a significantly large plurality of electrodes 110 to be accurate and repeatable positioned on the subject's leg 12. The electrodes 110 may be dual purpose, i.e., allowing for concurrent EMG recording and FES delivery, which makes the sleeve 100 well suited to sense motor intention and provide an intervention. Alternatively, the electrodes 110 may include receive and transmit electrode subsets for reading EMG and applying FES, respectively. In some suitable embodiments, custom software, for example, running on a laptop computer or the like, executes real-time or near real-time EMG decoding algorithms, interfaces the various system components, and allows for adjustments of the EMG and FES parameters. In some suitable embodiments, the system as disclosed provides a suitable solution to address FoG in patients with PD.

In general, some suitable embodiments disclosed herein provide an assistive technology in the form of a wearable, lower-limb sleeve 100 for concurrent recording of muscle activity (e.g., via EMG measurement) and electrical stimulation (e.g., such as FES, NMES or the like) of the affected muscles when a PD patient experiences a freezing episode. The sleeve 100 may be constructed of a lightweight, stretchable fabric that conforms to the patient's leg 12, e.g., similar to a compression sleeve. In some embodiments, the sleeve 100 may be worn around the lower portion of the leg (i.e., around the calf muscles), and a suitable hook and loop fastener or the like may be used to fasten opposing ends or sides of the sleeve 100 to one another, for example, longitudinally along the tibia. In practice, this may provide for a simple, streamlined method of donning and/or doffing. Advantageously, placement of hook and loop or other fastening mechanism along the tibia reduces variability in the EMG data by ensuring that the sleeve is consistently placed across subjects and sessions. In alternate embodiments, buttons, snaps, zipper, hook and eye or other suitable fastener may be used to selectively join the opposing ends or sides of the sleeve 100.

The high-density array of electrodes 110 embedded throughout the entirety of the sleeve 100 can permit simultaneous EMG and FES. Advantageously, combining EMG and FES capability into the same electrodes 110 allows for a smaller form factor and expanded spatial resolution across the leg 12 for recording and stimulation. Suitably, the sleeve's high-density array of electrodes 110 will enable extraction of spatial features across several muscles simultaneously. This feature provides a particular advantage, as PD patients can exhibit altered patterns of neuromuscular recruitment compared to healthy individuals. Additionally, as shown, the sleeve 100 may contain or incorporate the IMU 120 to measure kinematics of the lower leg to rapidly decode gait cycle phases for recognizing and/or determining the appropriate muscles to target with FES (e.g., stimulation of the gastrocnemius muscle during stance phase).

Generally, the sleeve 100 and accompanying system has the potential to improve FoG in PD patients. In suitable embodiments, the sleeve 100 is slim fitting, yet comfortable. As motion artifacts can contaminate EMG signals, motion of the electrode-skin interface can be minimized or reduced by providing a sufficiently snug fit of the sleeve 100 about the subject's leg 12 so that the electrodes 110 make good and consistent contact with the skin of the leg 12 under sufficient pressure applied by the sleeve 100 constricting about the leg 12. However, to help ensure it is comfortable and can be worn for an extended period, the fit is not overly snug or constricting. Suitably, the sleeve 100 is also sufficiently easy to don and doff. In some suitable embodiments, the sleeve 100 will fasten around the lower portion of the leg 12 between the ankle and knee, and may be donned and/or doffed by the patient or subject 10 without assistance. In practice, the sleeve 100 does not overly inhibit or restrict normal movement of the subject 10 or the subject's leg 12. Suitably, the sleeve 100 does not overly constrict the patient's range of motion and allows them to walk largely unencumbered.

In some suitable embodiments, detection of freezing episodes is achieved via recognition and/or identification of altered EMG patterns based on EMG data collected from the electrodes 110. Generally, surface EMG is a simple, non-invasive method to measure the electrical activity produced during a muscle contraction. Normal human walking produces a relatively cyclical pattern of muscle activity in the lower limb muscles. However, this pattern is altered immediately leading up to and during a freezing event and can be detected using the electrodes 110 placed on, near and/or adjacent to specific lower limb muscles. In some suitable embodiments, these abnormal patterns of muscle activity are leveraged or otherwise employed as a method to identify the onset of a freezing episode.

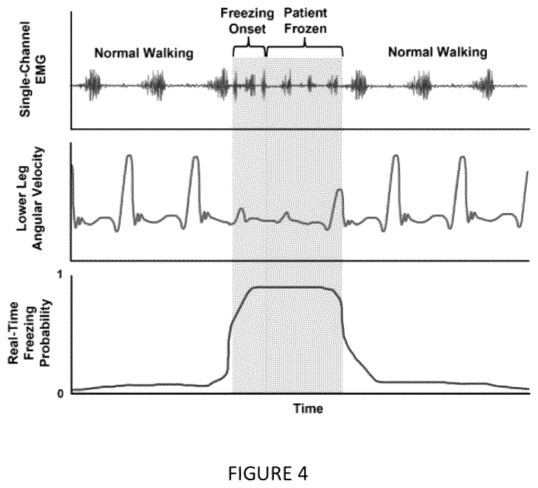

FIG. 4 shows a hypothetical case of single-channel EMG data collected from the leg of a PD patient who experiences a freezing episode. As can be seen from the EMG signal illustrated in the upper graph, normal, steady-state walking involves a cyclical pattern of lower limb muscle activity and an otherwise regular EMG signal produced thereby is represented at the left and right ends of the graph. However, immediately before the onset of a freezing episode, this pattern is disrupted, e.g., as represented by EMG signal at the portion of the graph labeled “Freezing Onset.” FIG. 4 also illustrates in the middle graph a determined angular velocity of the lower leg, e.g., measured with the aid of the embedded IMU 120 in the sleeve 100. This allows for accurate detection of gait events (e.g., heel strike, toe off, etc,) to determine the disrupted gait cycle phase which corresponds in time to the detected freezing onset. The bottom graph of FIG. 1 illustrates a hypothetical real-time or near real-time classification of altered muscle activity during a freezing episode.

In some suitable embodiments, a real-time or near real-time decoding algorithm may be used to sense, detect and/or other determine freezing onset base on the EMG activity in the subject's leg 12, for example, when a sufficient deviation is recognized and/or identified from EMG activity produced by an otherwise normal, healthy gait. In short, the real-time or near-real time EMG activity picked-up by the electrodes 110 can provide closed-loop control of the assistive technology, i.e., FES, NMES and/or the like administer by the same electrodes 110.

In some suitable embodiments, the controller 200 and/or decoder 220 implements a FoG detection algorithm to detect the onset of a freezing episode from PD patients' lower-limb muscle activity. In practice, the high-density array of electrodes 110 in the leg sleeve 100 may capture (in real-time or near real-time) muscle activity from the major muscles of the lower leg that are active during gait, for example, including the tibialis anterior, soleus, and gastrocnemius muscles. In some suitable embodiments, using these data, a neural network machine learning model may be trained to identify changes in the normal pattern of lower limb muscle activity and classify these alterations as a freezing episode. The embedded IMU 120 may capture the angular velocity of the shank during walking. In some suitable embodiments, by applying a wavelet transformation to these data, specific timings of heel strike and toe off can be extracted, thus allowing identification of the phase of the gait cycle (e.g., stance or swing) in which the freezing episode was detected. Accordingly, the gait cycle phase can be used to determine which group of muscles should be targeted with electrical stimulation.

In some suitable embodiments, to develop and/or refine the FoG detection algorithm employed by the controller 200 and/or decoder 220, EMG activity can be recorded from the lower leg in a group of test participants with PD who currently experience regular FoG episodes. As freezing episodes may be detectable from either leg of a PD patient, subjects and/or participants may don a sleeve on each leg for simultaneous bilateral measurement during walking, thereby allowing identification and/or understanding of any potential inter-limb differences during a freezing episode. Suitably, each test participant may complete a number (e.g., two or more) of sessions of test data collection. During each session, participants may perform a series of short, repeated walking trials, e.g., on a suitable obstacle course or the like. In practice, these trials can be designed to induce several freezing episodes by having the patient encounter common scenarios that trigger FoG. During each walking trials, bilateral, high-density EMG can be recorded from each participant's lower leg, as well as synchronized video of each trial. Accordingly, by reviewing each video and marking the onset of any freezing episodes, the time-referenced EMG data can be classified as representative of freezing. Suitably, the training trials can include other common scenarios, such as gait initiation and volitional stopping, throughout the session to produce a more robust FoG detection algorithm that can distinguish normal movement from freezing. Participant's subsequent visits can be used to assess the performance of personalized algorithms to identify freezing episodes as participants once again navigate the obstacle course. Additional sessions may be conducted to help to ensure algorithm robustness.

From the set of EMG test or trail data collected during each participant's training or experimental session, a machine learning algorithm or the like for real-time or near real-time decoding of FoG may be developed. In some suitable embodiments, time-domain features extracted from the high-density EMG signals collected via the electrodes 110 may be used to train a neural network classifier or the like, e.g., implemented in and/or by the controller 200 and/or the decoder 220. In some embodiments, alternative to a neural network classifier may be employed, e.g., such as logistic regression and/or support vector machine models. However, while potentially sufficient, a neural network may outperform other classifiers.

In some suitable embodiments, an individualized decoder may be developed, programmed, tuned and/or other provisioned for each patient or subject 10. Alternatively, a generalized decoder may be developed, programmed and/or provisioned that could be applied to several patients or subjects.

Given the dynamic nature of the proposed EMG measurements, motion artifacts could potentially contaminate the data and reduce the ability to effectively decode FoG with sufficient accuracy. Accordingly, in some embodiments, suitable data processing may be applied (e.g., by the controller 200 and/or decoder 210) to remove motion artifacts from high-density EMG recordings of the lower limb or leg 12 so that motion artifact content is substantially reduced, while retaining the otherwise true myoelectric activity. For example, in some suitable embodiments, the high-density EMG data from each participant can be cleaned using a multivariate signal processing approach, and the resulting high-fidelity EMG signals can be input into the neural network or decoder 220 to improve performance. In some suitable embodiments, freezing episodes with an F1 score greater than 0.9 can be detected, indicating achievement of a high rate of identifying true freezing episodes, while minimizing instances of falsely classified events as freezing episodes. Generally, the F1 score captures the precision and recall of the neural network and is a suitable metric given the relatively low incidence of freezing episodes relative to steady-state walking. Suitably, the real-time or near real-time decoding algorithm can identify freezing episodes within about 1.0 second (s) or less of onset, and an electrical stimulus (e.g., FES or NMES or the like) can be administer to the affected or selectively targeted muscles via the electrodes 110 within one second or less, and in some embodiments within about 300 milliseconds (ms) or less, of the detection of a freezing episode.

As disclosed herein, according to some suitable embodiments, FES, NMES or the like is administered to one or more selected target muscles or muscle groups of the leg 12 via corresponding electrodes 110 in the sleeve 110 to overcome and/or inhibit freezing episodes, e.g., associated with FoG in PD patients. That is to say, having successfully developed, tuned and/or otherwise implemented an algorithm or logic or the like (e.g., with the controller 200 and/or decoder 220) that recognized, detects and/or identifies (in real-time or near real-time) FoG episodes (e.g., based on EMG data obtained from the electrodes 110 in the sleeve 100), suitable stimulation is applied to effectively intervene when the onset of a freezing episode is detected. To this end, FES, NMES or like electrical or other effective stimulation is delivered one or more selected target muscles or muscle groups of the affected lower limb or leg 12. Generally, FES is a method of providing a mild electrical current to aid in the contraction of a target muscle or group of muscles. In some suitable embodiments, an electrical stimulus can be delivered via the electrodes 110 within one second or less, and in some embodiments within about 300 ms or less, of the detection of a freezing episode, i.e., well before the subject 10 has substantially frozen.

Generally, electrical stimulation may be an effective method for alleviating FoG symptoms. In some cases, patients who receive suitable stimulation, e.g., over a period of eight weeks, to the common peroneal nerve may be able to walk farther with a greater average stride length during a timed task. In a similar manner, FES applied during the swing phase of the gait cycle has the potential to decrease stride-to-stride variability and reduced the amount of time the patient spent in the double support phase of the gait cycle. Low-level sensory electrical stimulation applied to the quadriceps and hamstring muscles in a rhythmic pattern during walking also has the potential to reduce PD patients' walking time and the number of FoG episodes experienced during some short walking tasks.

As disclosed herein, according to some embodiments, FES, NMES or the like is used in concert with a real-time or near real-time EMG decoding algorithm or the like to overcome and/or inhibit FoG, via a closed-loop, EMG-driven delivery of corrective stimulation to one or more selected target muscles or muscle groups affected in the lower limb or leg 123 of the subject 10, immediately at or substantially near the time of onset of a freezing episode.

In general, many cue-based technologies operate after the patient has already frozen, or they continuously deliver a stimulus at fixed intervals, while some other methods trigger intervened based on changes in simple gait parameters, which may not represent the true neuromuscular alterations that occur when a patient experiences a FoG episode. Conversely, in accordance with some embodiments disclosed herein, FES, NMES or other like stimulation delivery can be based on biological data (e.g., EMG data) representative of altered neural control of movement. Suitably, the applied stimulation (e.g., FES) triggers action potentials in the afferent and efferent pathways that activate interneurons whose signals reach the motor cortex. Suitably, the repetitive application of volitionally controlled FES may encourage plasticity and lead to significant recovery of function in individuals with neurological disorders. Therefore, repeated use of EMG-driven FES may have the potential to reduce the number of freezing episodes a PD patient experiences both during and long after use of the device and/or system.

In some suitable embodiments, delivery of FES, NMES or the like during freezing episodes may be tested and/or tuned for PD patients who frequently experience FoG. For example, each participant or subject 10 may attend a baseline tuning session where the stimulation parameters (e.g., stimulation duration, waveform, pulse width, amplitude, etc. of an electrical stimulation delivered via the electrodes 110) will be tuned to ensure tolerability and comfort during walking. After the appropriate initial stimulation parameters are determined, participants may return for a subsequent session, during which they may receive FES to their affected muscles when a freezing episode occurs. Suitably, information gathered from the EMG decoding pilot testing may be used to determine if stimulation is provided to one or both of the patient's legs during walking. Participants may perform a series of walking tasks on the obstacle course while a physical therapist or other suitable individual accompanies them, e.g., to mitigate the risk of falls. In some suitable embodiments, during the tuning or trial sessions, a clinician or other individual may selectively deliver FES to the participant's leg 12, e.g., by pressing a button or other manual activation, as soon as they identify the onset of a freezing episode. Suitable, intended outcome measures can be used to establish and/or tune the stimulation parameters so that one or more selective target muscles and/or muscles groups of the lower-limb or leg 12 of the subject is safely and/or effectively stimulated during freezing episodes.

Several environmental and situational factors may increase the chance of a freezing occurrence. For example, gait initiation, approaching an object or destination, approaching and/or entering doorways, and multi-tasking may increase the likelihood of a freezing episode. Accordingly, to help ensure that the FoG detection algorithm and stimulation methods used in accordance with some embodiments herein are robust across a range of common everyday scenarios, the training, establishment and/or tuning employed may use the pair in tandem to identify FoG and intervene. In some suitable embodiments, in addition to measuring EMG activity from the lower limb of each participant or subject 10, a set of external IMUs may be placed on the trunk, arms, and legs to analyze several balance and gait parameters that are often altered in PD patients who experience FoG (e.g., stride length, postural sway, etc.).

During the test or train sessions, data collection may occur on a suitable obstacle course. In practice, subjects may attend two sessions, e.g., including a baseline session and an experimental session. During the baseline session, patients will don the EMG-FES leg sleeve 100 and complete a set of walking trials on the obstacle course while EMG data is recorded. Suitably, no stimulation will be delivered during these trials. These data can then be used to calibrate a personalized decoder 220 for real-time or near real-time identification of freezing episodes. Patients may also complete a number of relatively short walking trials in a wide-open area (i.e., an area not likely to induce freezing) to characterize each patient's normal, unaffected gait. After completing these trials, the FES parameters can be established and/or tuned for each patient to their level of comfort. Subsequently, patients may return for their experimental session to test the closed-loop EMG-FES system. Patients can once again complete walking trials on the obstacle course while wearing the leg sleeve 100. During this experimental session, stimulation can be delivered when their personalized decoder 220 detects the onset of a freezing episode. In some embodiments, using the angular velocity from the sleeve's embedded IMU 120, the phase of the gait cycle (e.g., stance, swing, etc.) that was affected by the FoG episode can be determined. Accordingly, stimulation and/or intervention can be coordinated to target one or more selected muscles or muscle groups based on when, relative to the gait cycle, the freezing is detected. For example, stimulation can be delivered to the ankle plantarflexor muscles (e.g., gastrocnemius) when freezing is detected to occur during the stance phase, while detected FoG during swing phase can trigger stimulation to the ankle dorsiflexor muscles (e.g., tibialis anterior).

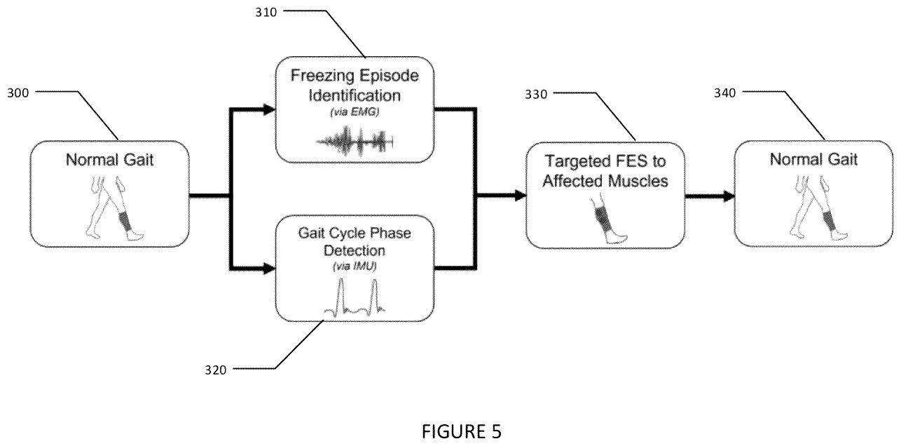

In accordance with some suitable embodiments disclosed herein, FIG. 5 diagrammatically depicts a method of coordinated FoG detection and triggered intervention/stimulation in response thereto. As shown in box 300, the subject 10 wearing the leg sleeve 100 on their leg 12 may be engaged in walking with a normal, healthy gait. Suitably, the controller 200 and/or decoder 220 may recognize and/or identify the gait as normal based on EMG data obtained from the electrodes 110 in the sleeve 100. As shown in box 310, when the subject 10 experiences the onset of a freezing episode, the controller 200 and/or decoder 220 may recognize and/or identify the same, e.g., in real-time or near real-time using the trained and/or otherwise established FoG detection algorithm or the like, based on EMG data obtained from the electrodes 110 in the sleeve 10. Coupled and/or in parallel therewith, as shown in box 320, the affected gait cycle phase may be detected and/or determined, e.g., based on data obtained from the IMU 120. Accordingly, as shown in box 330, the stimulator 210 may be triggered and/or controlled (e.g., by the controller 200) to selectively administer target FES, NMES or the like by energizing one or more selected electrodes 110 of the sleeve 100, e.g., which correspond to muscles or muscle groups of the leg 12 that are affected by the FoG episode. Accordingly, as shown in box 340, the one or more corresponding muscles or muscle groups so stimulated may be induced and/or urged into action such that the subject 10 remains walking uninterrupted, e.g., continuing normally with the appropriate phase of the gait cycle.

In practice, suitable outcome measures may be used to determine the safety and/or efficacy of the combined EMG-FES system disclosed herein, and may be used to further refine and/or tune various components and/or elements. For example, safety can be measured based on the number of reported adverse events, and efficacy can be determined based on the number of session trials and/or sessions successfully completed, as well as the number of successful freezing episodes detected and/or overcome with the stimulation delivered. For example, efficacy can be measured by the difference in the percentage of freezing episodes disrupted by the EMG-FES system compared to a control condition with no intervention (i.e., a control baseline).

PD patients who consistently experience FoG can have a lower quality of life, which may reflect the loss of control, decreased stability, and risk of falling that can occur due to freezing. Physical activity can have several therapeutic benefits for PD patients, including slower symptom progression and reduced cognitive decline. Regular exercise, e.g., combined with levodopa treatment can produce a synergistic effect on long-term motor recovery. However, some PD patients who experience regular freezing episodes develop a fear of falling, which can lead to a more sedentary lifestyle and greater withdrawal from society. Accordingly, reducing the severity and incidence of FoG via the closed-loop process disclosed herein can alleviate frustrating and stressful occurrences of freezing, and by improving mobility and the ability to be more physically active, PD patients may experience substantial improvements in their mental health and overall quality of life.

Though identified herein as a symptom of PD, FoG can be observed with other neurological conditions as well, and employment of some embodiments disclosed herein is likewise suitably applicable to some of these other neurological conditions. For example, individuals with muscular system atrophy and progressive supranuclear palsy can experience freezing episodes that disrupt their normal gait. Accordingly, a wider population (e.g., other than just PD sufferers) may benefit from embodiments disclosed herein to alleviate their symptoms. Though some embodiments herein are described with reference to an effective intervention within the PD community, some embodiments may have wide-reaching benefits across a multitude of neurological disorders that can affect an individual's gait, e.g., such as multiple sclerosis, stroke, and cerebral palsy. Many of these patients experience long-lasting walking impairments that similarly reduce their quality of life. Accordingly, the assistive technology disclosed herein may be expanded to improve the mobility and health outcomes in these clinical populations. Another potential benefit of the EMG-FES leg sleeve 100 and/or system disclosed herein is its ability to report on the activation of potential EMG-based biomarkers, thereby improving a current understanding of FoG manifestation. That is to say, in the aggregate, the high-density EMG data collected across a range of subjects may provide invaluable information that can be used to develop more effective, personalized treatment and rehabilitation strategies for PD patients.

Although a significant amount of research has sought to reduce the incidence of FoG in PD sufferers, heretofore, suitable methods have not been found which are sufficiently effective and/or consistent at identifying and overcoming freezing episodes across a broad spectrum of PD patients. For example, the effectiveness of pharmacological treatments can be viewed as inconclusive and can confound the incidence of freezing episodes. As well, other conventional and/or previously developed non-pharmacological treatments can suffer from a similar lack of effectiveness, due at least in part because they may not be suitably driven by instantaneous, real-time or near-real time changes in the patient's neuromuscular recruitment. Furthermore, these other conventional and/or previously developed approaches may not induce long-lasting benefits, and may lead to habituation, further decreasing their effectiveness. Conversely, to alleviate the detrimental effects of FoG, a solution is provided herein, in accordance with some suitable embodiments, to automatically and accurately identify when a freezing episode is imminent and/or onset, and then automatically administer corrective stimulation in sufficient time to overcome it.

In accordance with some embodiments, the approach disclosed herein suitably addresses both detection and intervention. For example, in the first instance, it provides automatic detection of FoG via high-density surface EMG measurements from several lower limb muscles that are active during gait. Surface EMG is non-invasive and easy to apply, thus making it a viable candidate for identifying perturbations resulting from a freezing episode. Second, when a freezing episode is detected from the patient's muscle activity, electrical stimulation can be instantaneously or readily delivered to the affected muscles, allowing the patient to continue walking normally. Moreover, the potential neuroplastic benefits of repeated FES may provide a possible long-term solution for those that suffer from FoG and/or PD.

There may be a risk that the lower limb sleeve 100 has a poor fit to the lower leg 12, e.g., in some PD patients. Accordingly, sleeves 100 may be manufactured and/or otherwise produced in a variety of different sized and/or shapes to accommodate subjects with legs of different sizes and/or shapes, e.g., sleeves 100 may be comfortable enough to be worn for an extended period across a range of PD patients. In some suitable embodiments, sleeves may be sized and/or shape to conform with the anthropometrics of the average PD patient. Suitably, feedback can be directly elicited from the PD patients who participate in the preliminary EMG decoding and FES data collections. This feedback can then be used to modify or adjust the size and/or shape of the sleeve 100, thereby helping to encourage patient compliance and inform future designs.

In some embodiments, a computational load due to simultaneous monitor, recording and/or processing of EMG data from both legs may provide challenges to real-time or near real-time EMG decoding of freezing episodes. Accordingly, to provide sufficient bandwidth to accommodate simultaneous capture of 100+ EMG channels coupled with online decoding, a wired connection may be used from each sleeve to a main data collection computer or device, e.g., such as the controller 200. In some suitable embodiments, a thin wire from each sleeve may run up the side of each participant's leg to their hip and may be tethered using surgical tape or the like. These wires can then connect to a nearby laptop or other like computer or data processing device. In some suitable embodiments, a wireless communication channel may be used with a sufficiently broad bandwidth to accommodate the data load. In some embodiments, the data load may be reduced by collecting data from each leg of the subject 10 independently and/or separately.

In some embodiments, data limitations due to infrequency and/or variability of FoG across patients may pose a challenge to decoding performance. Accordingly, the obstacle course employed in the training sessions may be arranged to allow data collection of FoG across a wide range of different scenarios. For example, the variety of obstacles provided may cover a wide range of common scenarios that trigger FoG, and accordingly, a robust testbed for high throughput data collection can be had. In some suitable embodiments, e.g., where standard supervised machine learning techniques may not perform suitably, alternative statistical methods, such as change point analysis and/or anomaly detection, may be employed as an alternative to or in combination with machine learning. Suitably, EMG frequency domain metrics may also be used to provide valuable information, and/or the sleeve's embedded IMU 120 may also provide additional information regarding real-time or near real-time changes in gait parameters that signal the onset of a freezing episode. In some suitable embodiments, these metrics may be combined to produce a more robust identification algorithm that leverages both the patient's muscle activity and lower limb kinematics.

The delivering of FES directly to the affected muscle groups can be an effective intervention among clinical populations whose neural pathways have been altered or damaged. In some embodiments disclosed herein, stimulation targets specific muscles that display altered patterns of EMG activity during the gait cycle. In some other embodiments, alternative stimulation sites, e.g., such as the peroneal and tibial nerves may be targeted. Suitably, the sleeve's high-density array of electrodes 110 allow for the exploration and/or utilization of various stimulation locations for effective delivery that can be customized to each subject.

In general, embodiments disclosed herein can advance treatment of FoG and improve PD patient outcomes by combining real-time or near real-time detection of freezing episodes prior to or at their onset with targeted, closed-loop stimulation to overcome them in a wearable sleeve 100.

In part, the present disclosure recognizes that normal lower limb muscle activity is altered before and during freezing in PD patients and uses that as an avenue for detecting FoG episodes. In essence, detection of that altered muscle activity (e.g., via EMG measurements or the like) is leveraged to identify FoG onset and timely trigger an intervention (e.g., suitably targeted FES, NMES or the like) that can effectively avert the freezing episode.

In the following, some further nonlimiting example methods and/or processes of detecting FoG and remediating the same are described. In these control systems, EMG collected from muscles of the legs (e.g., using the electrodes 110) is used to characterize impairment and train algorithms, The patient's biomechanics and muscle activation patterns during an average gait cycle are estimated, as well as the initiation and cessation of gait. These data are used along with biomechanical models and/or other sensorized analysis of gait to adjust NMES delivery scheme. During walking, the phase of the gait cycle is continuously estimated, the appropriate muscle activity is determined, and NMES is applied to improve gait.

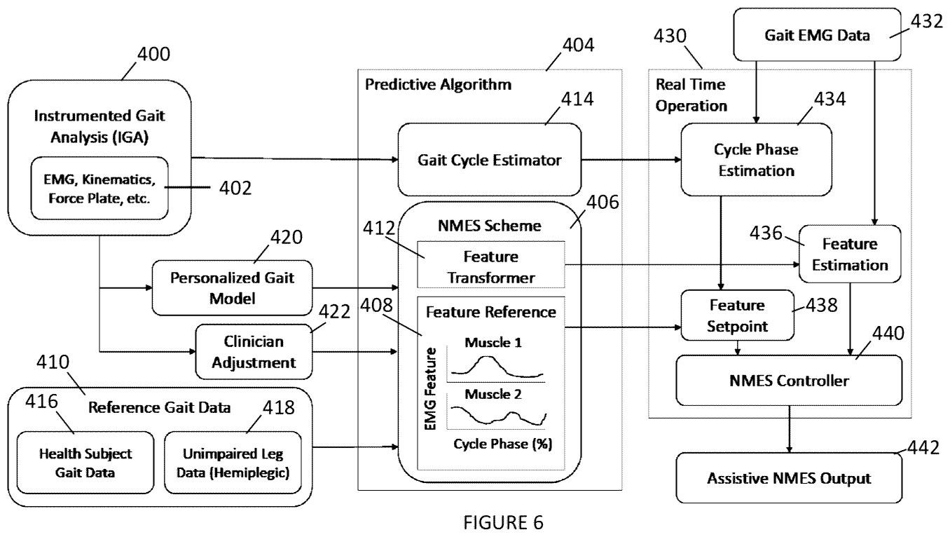

With reference now to FIG. 6, a further illustrative method and/or process of detecting FoG and remediating the same is shown by way of a flowchart. In an instrumented gait analysis (IGA) 400, data are collected from sensors 402 and analyzed. The sensors 402 may include EMG data from the legs collected using electrodes 110 (see FIGS. 2 and 3) while the user walks without NMES assistance. Additional sensors may include IMUs 120 (see FIGS. 2 and 3) collecting kinematic data from motion capture, or so forth. Collection of data additional to EMG data may increase performance and/or enable EMG data to be labeled with additional information, The IGA analysis 400 produces a predictive algorithm 404 for implementing an NMES assistance scheme 406. In the illustrative example NMES scheme 406, the data are decomposed into relevant features 408, such as biomarkers, multi-modal metrics, or so forth, and/or compared to data 410 of healthy reference subjects for clinician review. In one approach, an EMG feature transformer 412 is trained, for example to perform motor unit (MU) decomposition to associate EMG data (or EMG features 408 derived therefrom) with specific motor units of the musculature.

The IGA 400 further provides an EMG-based gait cycle estimator 414 that is trained from EMG data and/or IMU data. Historical data, if available, may be used to enhance performance and robustness of the gait cycle estimator 414 in low confidence situations such as the beginning and end of walking. In the predictive model 404 for NMES delivery, data from the IGA 400 and healthy data 410 if available (e.g., healthy subject gait data 416, unimpaired leg data 418 in the case of a hemiplegic subject, and/or so forth) is used to recommend an appropriate assistive NMES in accordance with the NMES scheme 406. Biomechanical models, e.g., a personalized gait model 420, may be used in conjunction with the EMG data to estimate EMG to kinematics relationship. Analysis may be reviewed by clinician via a user interface 422, who makes adjustments to NMES scheme.

A real time operation phase 430 operates on real-time EMG data 432 (and real-time IMU data if available, not shown in FIG. 6) to provide adjustable NMES to assist in the gait cycle and remediate gait freeze may operate as follows. Gait cycle phase estimation 434 is performed based on real-time EMG data 432 using the gait cycle estimator 414. From the real-time EMG data 432, feature values 422 are estimated 436 for each muscle, using the NMES scheme 406. Values are compared to the setpoint value 438 per the NMES scheme 406. During the real-time operation 430, a controller 440 adjusts NMES intensity 442 applied by the stimulator 210 (see FIG. 3) for each target muscle. The controller 440 may be a proportional-integral-derivative (PID) controller, a reinforcement learning (RL) controller, a model predictive control (MPC) controller, or so forth.

Closed-loop systems using sensors with poor estimation of gait, such as footswitches and tilt sensors, only provide data of a single joint or gait cycle events. By contrast, with EMG regression models 420 can be built to continuously estimate the progression through the cycle estimated by the gait cycle estimator 414. The EMG also precedes movement, meaning that gait errors can be detected earlier and predicted sooner. This allows for more responsive NMES, especially at the beginning and end of walking, which can be challenging to estimate. This sensorized approach to NMES delivery is optionally supplemented by the human-in-the-loop input provided by the clinician interface 422. This advantageously balances the automated and reproducible sensor control and the more personalized clinician control. Clinicians utilizing the interface 422 optionally provide expertise for therapy and can adjust the system in response to user feedback. By combining this EMG control scheme with analysis techniques and biomechanical modeling, a personalized assistive device is achieved. Biomechanical modeling software tools, such as OpenSim, are suitably used to predict the kinematics and muscle force of the user in response to activation (natural or stimulated) of muscles. These forces can further be estimated by EMG, for example using motor unit (MU) decomposition, a technique which improves prediction of force compared with EMG features that are not decomposed and associated to specific muscle units. Still further, EMG is an indicator of volitional intention, and pairing intention with NMES may provide more functional improvement. EMG control provides an embedded mechanism for challenge scaling, which can promote improvement in motor skill learning. As appropriate EMG is conditioned with this system, less assistive NMES is applied, creating continued challenge for the user. This may reduce the clinician burden for manual scaling.

Thus, EMG provides deep insight into the neuromotor coordination and intention without invasive sensors. Moreover, motor unit decomposition provides effective estimation of muscle force and neural control over activation. The high-density array of recording electrodes 110 facilitates this approach. Bidirectional NMES stimulation and EMG recording using the high-definition array of electrodes 110 of the sleeve 100 thus enables the disclosed FoG remediation scheme.

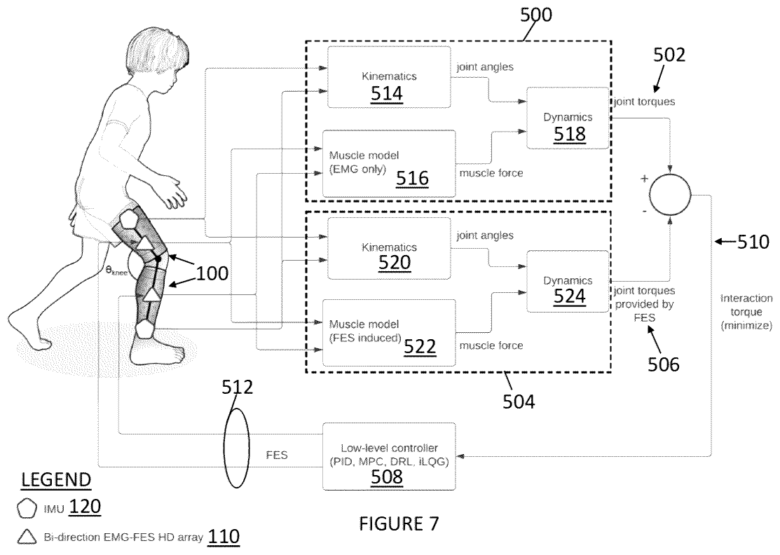

With reference now to FIG. 7, in some further nonlimiting illustrative embodiments, a closed-loop control approach employing wearable sensors such as the illustrative EMG (via electrodes arrays 110) and inertial IMU sensors 120 is used to calculate joint torque for use as a user-specific control signal. This reduces setup time, complexity and cost of personalized FES devices. In the illustrative example of FIG. 7, the sleeve 100 includes a first sleeve portion covering the thigh or upper leg and a second sleeve portion covering the calf or lower leg. This design provides more mobility for the knee which is uncovered. The illustrative control approach of FIG. 7 drives muscle activation towards goals derived from personal (user) data.

In one approach, IMU and EMG data are used to inform two neuromusculoskeletal models (e.g. Hill muscle model): a first neuromusculoskeletal model 500 for estimation of one or more current joint torques 502 (where the joint or joints may comprise a knee joint, an ankle joint, and/or so forth) to inform a required torque for compensation, and a second neuromusculoskeletal model 504 for estimation of the assistance torque 506 as a result of FES (given current joint positions). FES is suitably controlled with a low-level controller 508 (e.g. PID, model-predictive, iterative linear-quadratic-Gaussian (iLQG), or reinforcement learning) minimizing the difference 510 between the required torque 502 and the predicted assistance torque 506. Thus, the goal of the FES-provided torque 512 is equal to the required torque 502 predicted by the model. For example, insufficient ankle torque causing foot drop is calculated by the first model 500, with the second model 504 predicting the associated FES to match that torque assisting ankle dorsiflexion. Since EMG is measured before physical movement onset, the required torque 502 can be predicted and compensated for prior events. The nonlimiting illustrative first neuromusculoskeletal model 500 includes a kinematics model 514 and a muscle model 516. A musculoskeletal dynamic model 518 utilizes the kinematic (e.g., joint angle) output of the kinematics model 514 and the muscle force predicted from the measured EMG by the muscle model 516 to estimate the joint torques 502. The nonlimiting illustrative second neuromusculoskeletal model 504 likewise includes a kinematics model 520 and a muscle model 522, and a musculoskeletal dynamic model 524 that utilizes the kinematic (e.g., joint angle) output of the kinematics model 520 and the muscle force predicted to be produced by the FES by the muscle model 522 to estimate the joint torques 506 provided by the FES.

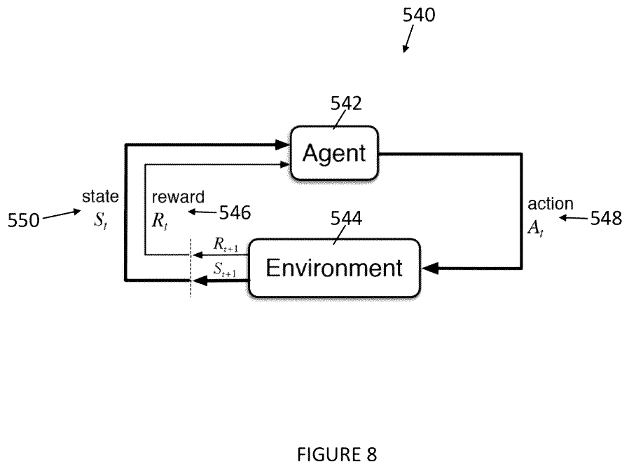

With reference to FIG. 8, another embodiment of a closed-loop control approach employing wearable sensors such as the illustrative EMG (via electrodes arrays 110) and inertial IMU sensors 120 to calculate joint torque for use as a user-specific control signal is described. Based on the IMU and EMG data and kinematic data collected in a calibration phase, a neuromusculoskeletal model of movement is tuned to an individual user, relating input signals, joint torques, and the kinematics of movement. In the embodiment of FIG. 8, a deep reinforcement learning (RL) algorithm 540 is used, including an agent 542 interacting with an environment 544. The RL algorithm 540 is used to optimize FES-driven torques for a metric of task success, such as energetic efficiency, which is known to be minimized in healthy trained motor tasks (e.g. walking). This metric is used as a reward signal 546 (having a value Rₜ at iteration t, and a value Rₜ₊₁ at iteration t+1) to update the policy employed by the RL agent 542 to select FES parameters representing an FES action (Aₜ) 548 that attempts to maximize the reward or task success 546. The FES action 548 may for example comprise FES timing, FES amplitude, FES location (i.e., which electrodes of the electrodes array 110 apply the FES), various combinations thereof, and/or so forth. For example, a negative reward of side-to-side gait sway will penalize the agent 542 for selecting FES actions that cause the sway. The policy of the agent 542 may for example be implemented using an artificial neural network (ANN), and the agent 542 can further include a critic that evaluates the selection of the action 548. The environment 544 is suitably represented by state variable 550 having a value Sₜ at iteration t and a value Sₜ₊₁ at iteration t+1. The state variable 550 can encode information such as joint angles, dynamics (joint torques determined from a musculoskeletal model based on input EMG and IMU data), and/or so forth. The goal of the agent 542 is suitably to maximize the reward 546, e.g., minimize sway and increase walking efficiency. Offline optimization through model simulation is a suitable first step, with online training optionally performed as well to tune the RL model 540 to experimental signals. The optional online training may in some embodiments be adaptive based on the resulting FES responses.

Compared to proportional FES schemes using EMG as a target, torque-based control such as that employed in the embodiments of FIGS. 7 and 8 inherently avoids counterproductive antagonistic stimulation. For example, if antagonist muscles are spastically co-activated (which can occur, for example, in stroke discoordination) EMG-driven FES would provide proportional stimulation to both muscles, whereas torque—driven FES would only stimulate the muscle effecting net torque. Offline reinforcement learning of FES parameters with musculoskeletal models is expected to substantially reduce the time for fine tuning parameters during operation.

While two examples of closed-loop control approaches employing wearable sensors such as the illustrative EMG (via electrodes arrays 110) and inertial IMU sensors 120 to calculate joint torque for use as a user-specific control signal are described with reference to FIGS. 7 and 8, other types of dynamic neuromusculoskeletal models, such as models employed in robotics-based control solutions, are also contemplated.

In various embodiments disclosed herein, closed-loop electrical stimulation is employed to correct each by either disrupting pathological activity or assisting normal muscle activation therefore gait. Events during gait are detected using inertial measurement units (IMUs), stretch sensors around muscles, electromyography (EMG) sensors, and/or so forth. This enables event-triggered ES to correct these disorders. Electrical stimulation of electrodes of the (preferably high density) array of electrodes 110 is adaptively adjusted to better correct future gait events based on feedback from previous events. Event-related electrical stimulation-enabled correction metric(s) is/are quantified through approximation from sensor feedback comparison to healthy data or relevant metric of gait (e.g. efficiency, symmetry, or duration of freezing) based on EMG and IMU recordings around and after the event. The metric is used to infer and incorporate context, such as walking surface incline or user fatigue. Based on a model of the electrical stimulation effecting movement (e.g. increased gastrocnemius stimulation effects increased foot plantarflexion), stimulation parameters, such as amplitude or duration, are updated for the event. In practice, this may take various forms such as ramping electrical stimulation amplitude at successive freezing of gait (FoG) events, prompting the user through haptic-level stimulation then assisting the user through neuromuscular stimulation to execute the next step, until an appropriate level of intervention is reached. Additionally, the stimulation can adapt to the context of FoG events, such as stimulating appropriate muscles to execute the next phase of gait as the algorithm learns to detect (for example) if FoG occurs before the left or right step.

Gait correction based on event detection which is static during operation (that is, after being set, each detected event generates the same stimulation sequence) may be sufficient for the user during the settings calibration; however, new contexts (e.g., speed, walking surface, changing electrode impedance) benefit from adjusted electrical stimulation as disclosed herein to maintain efficacy. Such adjustment estimates the efficacy of previous stimulation through EMG and IMU feedback to adapt to these contexts. Advantageously, the EMG and IMU sensors are readily integrated into the wearable sleeve 100. Furthermore, the disclosed approaches can employ a variety of success metrics, such as gait efficiency, symmetry, speed, regularity, or normality of muscle activation readily computed from EMG and FES sensors. These metrics capture a more holistic view of the effects of stimulation to better tailor parameters. For example, if stimulation caused an overextended step, the next step may be inappropriately disrupted. Holistic measurements employed herein enable adjustment of parameters to reduce the overextension, whereas a simple metric like ankle joint angle would be insensitive to the disruption.

The above methods, system, modules, units, processes, algorithms, devices and/or apparatus have been described with respect to particular embodiments. It is to be appreciated, however, that certain modifications and/or alteration are also contemplated.

It is to be appreciated that in connection with the particular exemplary embodiment(s) presented herein certain structural and/or function features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

It is also to be appreciated that any one or more of the particular tasks, steps, processes, methods, functions, elements and/or components described herein may suitably be implemented via hardware, software, firmware or a combination thereof. In particular, various modules, components and/or elements may be embodied by processors, electrical circuits, computers and/or other electronic data processing devices that are configured and/or otherwise provisioned to perform one or more of the tasks, steps, processes, methods and/or functions described herein. For example, a processor, computer, server or other electronic data processing device embodying a particular element may be provided, supplied and/or programmed with a suitable listing of code (e.g., such as source code, interpretive code, object code, directly executable code, and so forth) or other like instructions or software or firmware, such that when run and/or executed by the computer or other electronic data processing device one or more of the tasks, steps, processes, methods and/or functions described herein are completed or otherwise performed. Suitably, the listing of code or other like instructions or software or firmware is implemented as and/or recorded, stored, contained or included in and/or on a non-transitory computer and/or machine readable storage medium or media so as to be providable to and/or executable by the computer or other electronic data processing device. For example, suitable storage mediums and/or media can include but are not limited to: floppy disks, flexible disks, hard disks, magnetic tape, or any other magnetic storage medium or media, CD-ROM, DVD, optical disks, or any other optical medium or media, a RAM, a ROM, a PROM, an EPROM, a FLASH-EPROM, or other memory or chip or cartridge, or any other tangible medium or media from which a computer or machine or electronic data processing device can read and use. In essence, as used herein, non-transitory computer-readable and/or machine-readable mediums and/or media comprise all computer-readable and/or machine-readable mediums and/or media except for a transitory, propagating signal.

Optionally, any one or more of the particular tasks, steps, processes, methods, functions, elements and/or components described herein may be implemented on and/or embodiment in one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device capable of implementing a finite state machine that is in turn capable of implementing the respective tasks, steps, processes, methods and/or functions described herein can be used.

Additionally, it is to be appreciated that certain elements described herein as incorporated together may under suitable circumstances be stand-alone elements or otherwise divided. Similarly, a plurality of particular functions described as being carried out by one particular element may be carried out by a plurality of distinct elements acting independently to carry out individual functions, or certain individual functions may be split-up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that all such modifications and alterations are included herein insofar as they come within the scope of the appended claims or the equivalents thereof. It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art, which are also intended to be encompassed by the following claims.

What is claimed is:

1. A device for overcoming freezing of gait (FoG), the device comprising:
a sleeve sized and shaped to be worn on a leg of a human subject;
an array of electrodes carried by the sleeve and positioned to make surface contact with skin of the leg when the sleeve is worn on by the human subject; and
a controller in communication with the array of electrodes, said controller being operative to:
receive muscle activity data from the array of electrodes indicative of muscle activity in the leg;
identify an onset of a FoG episode based on the received muscle activity data; and
trigger energization of one or more targeted electrodes in response to identification of the onset of the FoG episode to mitigate against the FoG episode.

2. The device of claim 1, wherein the muscle activity data comprises electromyography (EMG) measurements obtained via the array of electrodes.

3. The device of claim 1, wherein the sleeve further includes an inertial measurement unit (IMU) that is responsive to movement of the leg.

4. The device of claim 3, wherein the controller is further operative to:
obtain motion data from the IMU indicative of movement of the leg;
determine a phase of a gait cycle the subject is in based on the obtained motion data; and
select one or more of the array of electrodes as the one or more target electrodes that are energized based on the determined phase of the gait cycle the subject is in.

5. The device of claim 1, wherein the controller comprises:
a decoder that processes the muscle activity data received from the electrodes to identify the onset of the FoG episode; and
a stimulator that provides an electrical signal to the one or more target electrodes to thereby energize the one or more target electrodes.

6. The device of claim 1, wherein the FoG episode is identified by detecting a deviation in the muscle activity data received from the electrodes from muscle activity data recognized as corresponding to a normal gait.

7. The device of claim 1, wherein the onset of the FoG episode is identified within less than or equal to 1 second from a time that the FoG episode begins to be onset.

8. The device of claim 1, wherein the energization of the one or more target electrodes occurs within less than or equal to one second from a time that the onset of the FoG episode is identified.

9. The device of claim 1, wherein the energization of the one or more target electrodes provides one of functional electrical stimulation (FES) and neuromuscular electrical stimulation (NMES) to one or more muscles of the leg corresponding to positions of the one or more target electrodes, such that the one or more muscles of the leg are induced to contract accordingly.

10. The device of claim 1, wherein the sleeve is stretchable and constricts about the leg when worn by the subject so that the array of electrodes are pressed against the skin of the leg.

11. An apparatus providing walking assistance for a subject with a neurological impairment, the apparatus comprising:
a sleeve arranged to be fitted on a leg of the subject;
an array of electrodes carried by the sleeve and positioned to make contact with a skin of the leg when the sleeve is fitted to the subject; and
at least one processor which executes computer program code from at least one memory, wherein the at least one memory and the computer program code are configured, with the at least one processor, to cause the apparatus at least to:
collect electromyography (EMG) data from the array of electrodes, the EMG data being indicative of muscle activity in the leg;
model the EMG data to determine assistive neuromuscular electrical stimulation (NMES) for improving a gait of the subject; and
trigger energization of one or more targeted electrodes in accordance with the determined assistive NMES.

12. The apparatus of claim 11, wherein the EMG data is modeled to determine the assistive NMES by operations including performing muscle unit (MU) decomposition of the EMG data.

13. The apparatus of claim 11, wherein the sleeve further includes an inertial measurement unit (IMU) that is responsive to movement of the leg and wherein the at least one memory and the computer program code are configured, with the at least one processor, to further cause the apparatus at least to:
obtain motion data from the IMU indicative of movement of the leg;
wherein the assistive NMES is further determined based on the motion data from the IMU.

14. The apparatus of claim 11, wherein the assistive NMES is further determined by estimating a phase of a gait cycle of the subject based on the EMG data and/or motion data from an inertial measurement unit (IMU) that is indicative of movement of the leg.

15. The apparatus of claim 11, wherein the at least one processor executing the computer program code from the at least one memory further provides a user interface via which a clinician can adjust the modeling of the EMG data to determine the assistive NMES.

16. The apparatus of claim 11, wherein the modeling of the EMG data to determine the assistive NMES for improving the gait of the subject includes modeling the EMG data to determine a joint torque for improving the gait.

17. The apparatus of claim 16, wherein the modeling of the EMG data to determine the joint torque for improving the gait comprises modeling using a reinforcement learning (RL) model.

18. A method for detecting and overcoming freezing of gait (FoG), the method comprising:
monitoring muscle activity of a leg of a subject with an array of electrodes positioned in contact with a skin of the leg, the array of electrodes being carried by a sleeve worn on the leg;
identifying an onset of a FoG episode based on the monitored muscle activity; and
selectively energizing of one or more targeted electrodes of the array of electrodes in response to identification of the onset of the FoG episode to remediate the FoG episode.

19. The method of claim 18, further comprising:
monitoring motion of the leg; and
determining a phase of the subject's gait within a gait cycle base at least in part on the monitored motion.

20. The method of claim 19, further comprising:
selecting one or more of the array of electrodes as the one or more target electrodes that are selectively energized based on the determined phase of the subject's gait.
